(19) United States
(12) Patent Application Publication
(10) Pub. No.: US 2003/0105428 A1
(43) Pub. Date: Jun. 5, 2003

(54) Electronically controlled fluid delivery device

(76) Inventors: John Hogan, Golden Valley, MN (US); Terry Bunge, Bunsville, MN (US)

Correspondence Address: Schwegman Lundberg Woessner & Kluth PA, P O Box 2938, Minneapolis, MN, 55402, US

(21) Appl. No.: 10/011,902

(22) Filed: Dec. 5, 2001

(51) Int. Cl. A61M 37/00

(52) U.S. Cl. 604/131; 604/246; 128/DIG. 12

(57) ABSTRACT

An apparatus for delivering fluids at a precisely controlled rate which comprises a fluid dispensing component having a fluid reservoir for containing the fluids to be delivered and an electrically operated flow control device operably associated with the fluid dispensing component. The electrically operated flow control device comprises a solenoid valve for precisely controlling the flow of medicinal fluids to the patient. More particularly, the solenoid valve functions to precisely control both the frequency of the flow of medicinal fluid toward the patient and the volume of medicinal fluid flowing toward the patient.

### BACKGROUND OF THE INVENTION

1. Field of the Invention

The present invention relates generally to fluid delivery devices. More particularly, the invention concerns an apparatus for delivering medicinal fluids to a patient that includes a fluid dispenser having a built-in stored energy means comprising at least one distendable elastomeric membrane and an electrically operated fluid flow control means operably associated with the fluid dispenser for controllably infusing medicinal agents into an ambulatory patient at specific rates over specific periods of time.

2. Discussion of the Invention

Many medicinal agents require an intravenous route for administration thus bypassing the digestive system and precluding degradation by the catalytic enzymes in the digestive tract and the liver. The use of more potent medications at elevated concentrations has also increased the need for accuracy in controlling the delivery of such drugs. The delivery device, while not an active pharmacologic agent, may enhance the activity of the drug by mediating its therapeutic effectiveness. Certain classes of new pharmacologic agents possess a very narrow range of therapeutic effectiveness, for instance, too small a dose results in no effect, while too great a dose results in toxic reaction.

In the past, prolonged infusion of fluids has generally been accomplished using gravity flow methods, which typically involve the use of intravenous administration sets and the familiar bottle suspended above the patient. Such methods are cumbersome, imprecise and require bed confinement of the patient. Periodic monitoring of the apparatus by the nurse or doctor is required to detect malfunctions of the infusion apparatus.

Devices from which liquid is expelled from a relatively thick-walled bladder by internal stresses within the distended bladder are well-known in the prior art. Such bladder, or “balloon” type, devices are described in U.S. Pat. No. 3,469,578, issued to Bierman and in U.S. Pat. No. 4,318,400, issued to Perry. The devices of the aforementioned patents also disclose the use of fluid flow restrictors external of the bladder for regulating the rate of fluid flow from the bladder.

The prior art bladder type infusion devices are not without drawbacks. Generally, because of the very nature of bladder or “balloon” configuration, the devices are unwieldy and are difficult and expensive to manufacture and use. Further, the devices are somewhat unreliable and their fluid discharge rates are frequently imprecise.

The apparatus of the present invention overcomes many of the drawbacks of the prior art by eliminating the bladder and making use of more recently developed elastomeric films and similar materials, which, in cooperation with a base define a fluid chamber that contains the fluid which is to be dispensed. The elastomeric film membrane controllably forces fluid within the chamber into fluid flow channels provided in the base.

The elastomeric film materials used in the apparatus of the present invention, as well as various alternate constructions of the fluid delivery apparatus, are described in detail in U.S. Pat. No. 5,205,820 issued to Kriesel. Therefore, U.S. Pat. No. 5,205,820 is hereby incorporated by reference in its entirety as though fully set forth herein. U.S. Pat. No. 5,721,382, issued to Kriesel et al, also describes various alternate constructions and modified physical embodiments of the present invention including the provision of a novel fluid actuated indicator means for visually indicating fluid flow from the device. This patent is also hereby incorporated by reference in its entirety as though fully set forth herein.

The apparatus of the present invention that uniquely includes a novel electrically operated fluid flow control means can be used with minimal professional assistance in an alternate health care environment, such as the home. By way of example, devices of the invention can be comfortably and conveniently removably affixed to the patient's clothing or to the patient's body and can be used for the continuous infusion of antibiotics, hormones, steroids, blood clotting agents, analgesics including morphine, and like medicinal agents. Similarly, the devices can be used for I-V chemotherapy and can accurately deliver fluids to the patient in precisely the correct quantities and at extended microfusion rates over time.

### SUMMARY OF THE INVENTION

It is an object of the present invention to provide an apparatus for delivering fluids at a precisely controlled rate which comprises a fluid dispensing component having a fluid reservoir for containing the fluids to be delivered and an electrically operated flow control means operably associated with the fluid dispensing component and comprising a solenoid valve for precisely controlling the flow of medicinal fluids to the patient.

Another object of the invention is to provide an apparatus as described in the preceding paragraph in which the electrically operated flow control means comprises an electrically operated solenoid valve that functions to precisely control both the frequency of the flow of medicinal fluid toward the patient and the volume of medicinal fluid flowing toward the patient. More particularly, the flow control means comprises a control circuit, including a complementary metal oxide semiconductor, a first rotatable control knob operably associated with the control circuit for setting a time for opening the solenoid and a second rotatable control knob operably associated with the control circuit for setting the time interval between the opening and closing of the solenoid valve.

Another object of the invention is to provide an apparatus of the aforementioned character that includes a reservoir fill component that can be used to controllably fill the reservoir of the dispensing component.

It is another object of the invention to provide an apparatus of the aforementioned character that is highly reliable and easy-to-use by lay persons in a non-hospital environment.

Another object of the invention is to provide an apparatus which can be factory prefilled with a wide variety of medicinal fluids or one which can readily be filled in the field shortly prior to use using the novel reservoir fill component which can be removably interconnected to the lower surface of the base of the fluid dispenser.

Another object of the invention is to provide a reservoir fill assembly as described in the preceding paragraph that includes a specially designed polarity collar that enables it to be mated only with specifically configured fluid dispenser assemblies thereby preventing the fluid dispenser reservoir from being filled with an improper medicament.

Another object of the invention is to provide an apparatus as defined in the preceding paragraph in which the reservoir fill assembly is uniquely designed to accept a vial component of conventional construction which is factory filled with the medicinal fluid to be delivered to the patient.

A further object of the invention is to provide an accurate and highly reliable fluid delivery device, which can be manufactured inexpensively in large volume by automated machinery.

Another object of the invention is to provide a novel reservoir fill assembly for use with the fluid dispenser subassembly of the apparatus which is easy to use, is inexpensive to manufacture, and one which maintains the container in a substantially aseptic condition until time of use.

Other objects of the invention will become more apparent from the discussion, which follows.

By way of summary, the fluid delivery apparatus of the present form of the invention comprises two principal cooperating assemblies, namely a fluid dispenser and an electrically operated flow control means operably associated with the fluid dispenser for precisely controlling the flow of medicinal fluid to the patient. The fluid dispenser of one form of the invention, which readily lends itself to automated manufacture, is generally similar to that described in U.S. Pat. No. 05,962,794 issued to Kriesel and includes a base and a stored energy means comprising at least one distendable elastomeric membrane which cooperates with the base to form a fluid reservoir. In one form of the invention, the apparatus includes a reservoir fill means for controllably filling the reservoir of the device.

### BRIEF DESCRIPTION OF THE DRAWINGS

FIG. 1 is a generally perspective, bottom view of one form of the fluid dispensing apparatus of the invention.

FIG. 2 is a generally perspective view of one form of the reservoir fill means of the invention for filling the reservoir of the fluid dispenser.

FIG. 3 is an enlarged bottom plan view of the apparatus shown in FIG. 1.

FIG. 4 is an enlarged, cross-sectional view taken along lines 4-4 of FIG. 3

FIG. 5 is an enlarged, cross-sectional view taken along lines 5-5 of FIG. 3 showing the fluid dispenser component of the invention operably mated with the form of reservoir fill assembly illustrated in FIG. 2.

FIG. 6 is a cross-sectional view taken along lines 6-6 of FIG. 5.

FIG. 7 is a generally schematic view of the apparatus shown in FIG. 1 illustrating a portion of the control means of the invention, including an electrically operated solenoid valve.

FIG. 8 is a generally perspective view of one form of control knob of the invention for controlling the frequency of operation of the solenoid valve.

FIG. 9 is a generally perspective view of another form of control knob of the invention for controlling the interval of time between dosages.

FIG. 10 is a generally schematic view of one form of the control circuits of the control means of the invention for controlling the flow of medicinal fluid to the patient.

FIG. 11 is a generally schematic view graphically illustrating the operation of the control means of the invention for controlling the flow of medicinal fluid to the patient.

FIG. 12 is a generally perspective, top view of an alternate embodiment of the fluid delivery apparatus of the invention showing a different form of reservoir fill assembly and indicating exemplary dosage amounts and time periods between dosage.

FIG. 13 is a generally perspective, exploded view of the forward portion of the fluid dispenser of the alternate embodiment of the fluid delivery apparatus of the invention shown in FIG. 12.

FIG. 14 is an enlarged front view of the fluid dispenser portion of the apparatus shown in FIG. 12.

FIG. 15 is a cross-sectional view taken along lines 15-15 of FIG. 14.

FIG. 16 is an enlarged, cross-sectional view taken along lines 16-16 of FIG. 15.

FIG. 17 is an enlarged, cross-sectional view of the area identified in FIG. 15 by the numeral 17.

FIG. 18 is a generally perspective, broken away view of the blunt end cannula component of the fluid dispenser of the alternate embodiment of the fluid delivery apparatus of the invention shown in FIGS. 15 and 17.

### DESCRIPTION OF THE INVENTION

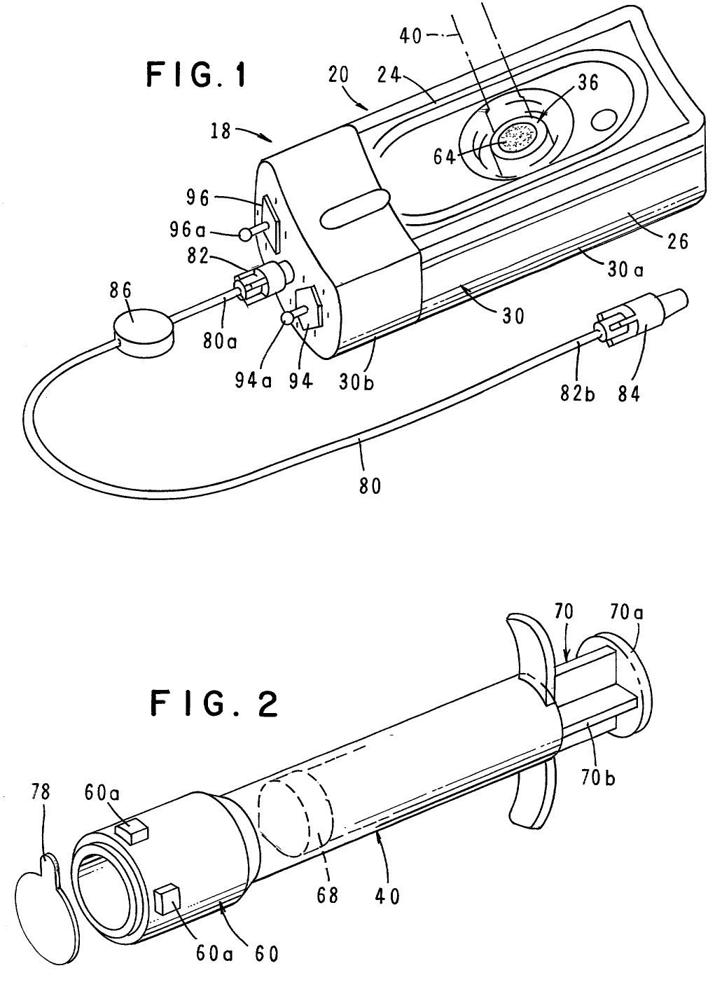

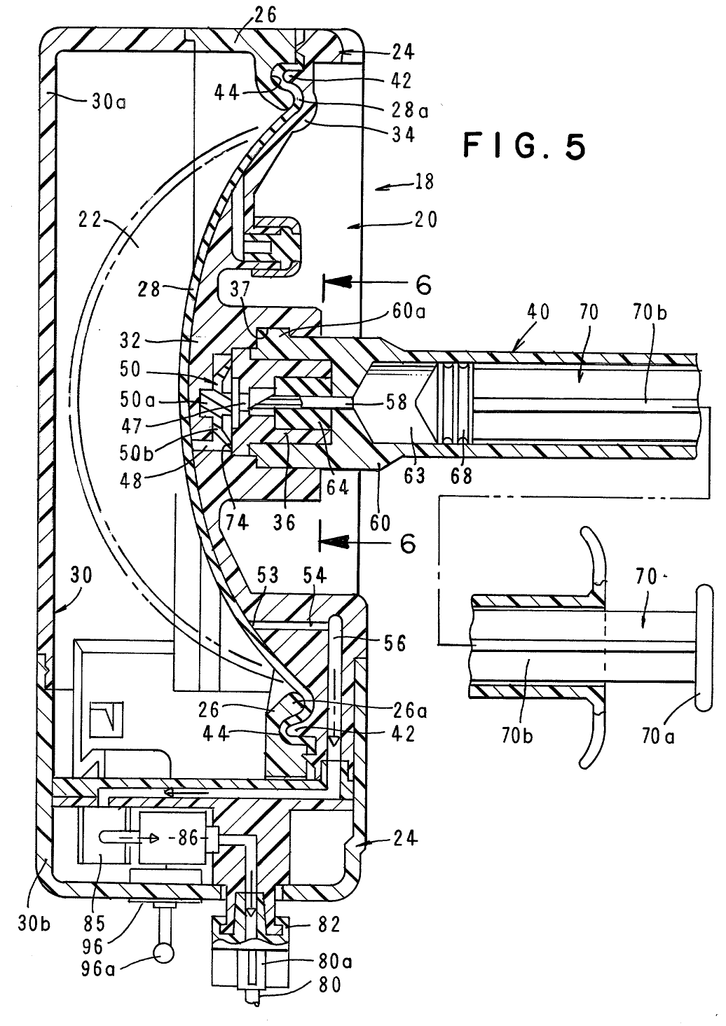

Referring to the drawings and particularly to FIGS. 1 through 5, one form of the apparatus of the invention is there illustrated and generally designated by the numeral 18. The apparatus here comprises three major cooperating components, namely a fluid dispensing apparatus or fluid dispenser 20, a reservoir fill means for filling the reservoir 22 of the fluid dispenser (FIG. 5) and an infusion means for controllably delivering medicinal fluids to the patient from the fluid dispenser.

Turning particularly to FIG. 4, the fluid dispenser 20 of the invention can be seen to include a housing assembly comprising a base 24, a capture ring 26, a stored energy source. Cover 30 includes a rear portion 30a and a forward cap-like portion 30b that, in a manner later to be described, houses the important flow control means of the invention for controlling fluid flow toward the patient. The base 24 includes an ullage defining protuberance 32 and a membrane capture portion 34. Disposed between base 24 and cover 30 is the previously mentioned membrane capture ring 26 that has a bottom opening 26a that receives protuberance 32 of base 24 (see FIG. 4).

Base 24 comprises, in addition to the distendable member engaging protuberance, or ullage 32, a dispenser connector subassembly 36, to which the reservoir fill assembly 40 is interconnected in the manner shown in FIG. 5. Base 24 also includes an upstanding tongue 42 that extends about the perimeter of the base and is closely receivable within a groove 44 formed in the capture ring 26 (FIG. 4). When the base 24 and the membrane capture ring 26 are assembled in the manner shown in FIG. 4, the periphery 28a of a distendable membrane 28 will be securely clamped within groove 44 by tongue 42. After the parts are thus assembled, base 24 is interconnected with capture ring 26 by any suitable means such as sonic bonding that also functions to simultaneously trim membrane 28. This done, cover 30 is mated with capture ring 26 in the manner shown in the drawings and is suitably bonded in place. Cover 30 can, if desired, be constructed from any suitable plastic material that is impermeable to fluids, including gases. Reference should be made to incorporated by reference U.S. Pat. No. 5,205,820 for a description of the various materials that can be used to construct the base 24, the capture ring 26, the cover 30 and the membrane 28.

During the reservoir filling step, the details of which will presently be described, fluid under pressure will flow into fluid inlet 47 of base 24, into passageway 48 of the fluid dispenser via an umbrella valve 50 and thence into reservoir 22 which is formed between protuberance 32 and distendable membrane 28. As the fluid under pressure flows into the reservoir, it will cause membrane 28 to distend outwardly from protuberance 32 in the manner shown by the phantom lines in FIG. 5. While the stored energy means can be in the form of a single prestressed or unstressed isotropic, elastomeric distendable membrane, such as membrane 28, it can also be constructed as a laminate assemblage made up of a plurality of initially generally planar distendable elements of films. Such construction is described in U.S. Pat. No. 5,721,382, which Patent is incorporated herein by reference. During the infusion step, internal stresses formed in membrane 28 will cause it to move toward protuberance 32 which will cause the fluid within reservoir 52 to be uniformly and controllably forced outwardly through an outlet 53, through a first passageway 54 and then through a second communicating passageway 56 formed in base 24 (FIG. 5) in a direction toward the infusion means of the invention.

As illustrated in FIG. 2, the reservoir fill means of the present embodiment of the invention comprises a syringe type fill assembly 40 that contains the medicinal fluid with which the reservoir of the dispensing apparatus is to be filled. A specially configured syringe barrel or polarity collar 60 functions to selectively interconnect the syringe assembly only with a medicament dispenser that is designed to delivery a particular medicament in a manner presently to be described so that fluid can be controllably transferred from the syringe to the reservoir of the dispenser component.

As best seen in FIG. 4, connector subassembly 36 includes a fluid chamber 62 having first and second open ends 62a and 62b. First open end 62a is sealably closed by closure means, here provided in the form of a pierceable septum 64. In the manner shown in FIG. 5, septum 64 is pierceable by the cannula means or cannula 58 of the syringe assembly. To expel fluid from fluid chamber 64 of the syringe assembly and into cannula 58 and thence into the fluid reservoir 22, a plunger 68 is provided. Plunger 68 is telescopically movable within chamber 64 by a pusher subassembly 70 that includes a pusher hub 70a and an elongated pusher stem 70b that is connected to the pusher hub FIG. 5.

Disposed within a chamber 72 that communicates with passageway 62 is the previously identified, umbrella check valve 50 that forms a part of the valve means of the invention for controlling fluid flow from cannula 58 toward the fluid reservoir of the dispenser assembly. Check valve 50, which is of a conventional construction, is designed to permit fluid flow toward reservoir 22, but to block fluid flow in the opposite direction. As shown in FIG. 5, check valve 50, includes a body portion 50a and a seat portion 50b that sealably engages seat 74 when the valve is in a closed position. The construction and operation of valve 50 is well understood by those skilled in the art and the manner of opening the valve during the filling step will presently be described.

Prior to use, a tear-away cap 76 is received over and closes the dispenser connector subassembly 36. Cap 76 maintains the dispenser connector and fluid inlet passageway of the device in a closed and substantially sterile condition. Similarly, a cap 78 maintains the syringe assembly in a closed condition until time of use.

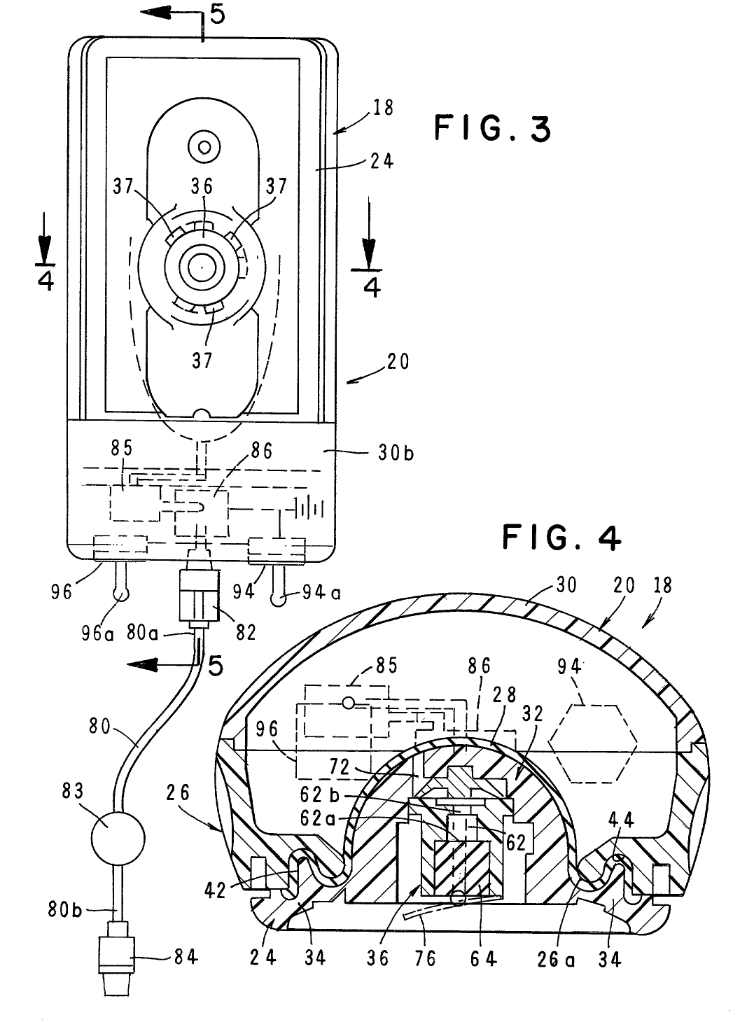

Turning now particularly to FIGS. 3 and 5, one form of the infusion means of the invention for delivering the medicinal fluid to the patient is there shown. The infusion means here comprises an elongated fluid delivery line 80 having a first end 80a connected to the outlet port luer type connector fitting 82 of housing 20 of the fluid dispenser and a second end 82b to which a conventional luer connector assembly 84 is connected (see also FIG. 1). Disposed intermediate the first and second ends of the fluid delivery line is a conventional gas vent assembly 86.

Also forming a part of the infusion means of the invention is filter means for filtering particulate matter from the medicinal fluids flowing toward the patient. The filter means comprises a conventional filter unit 85 of a character well known in the art, the filter element of which can be constructed from a number of porous materials such as metal and ceramics. A polyester sulfone material sold by Gelman Sciences under the name and style “SUPOR” has also proven satisfactory. As shown in FIG. 5, filter assembly 85 is housed within the forward portion 30b of the dispenser housing. Also housed within forward portion 30b is the electrically operated flow control means of the invention which functions to precisely control the flow of medicinal fluid toward the patient. As will presently be described, this novel electrically operated flow control means controls both the frequency of the flow of medicinal fluid toward the patient as well as the volume of medicinal fluid flowing toward the patient.

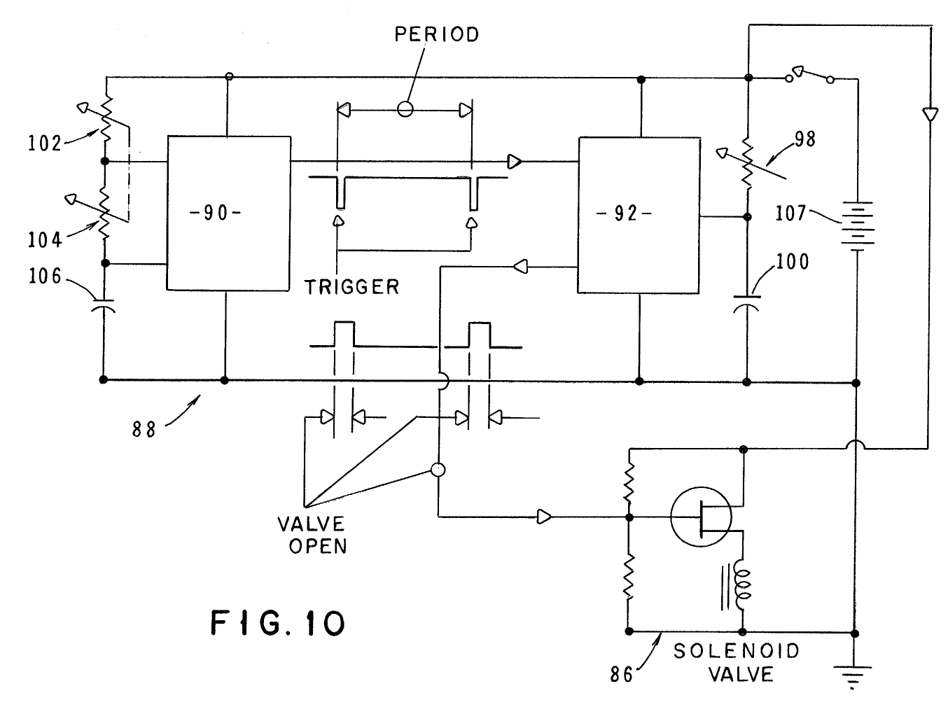

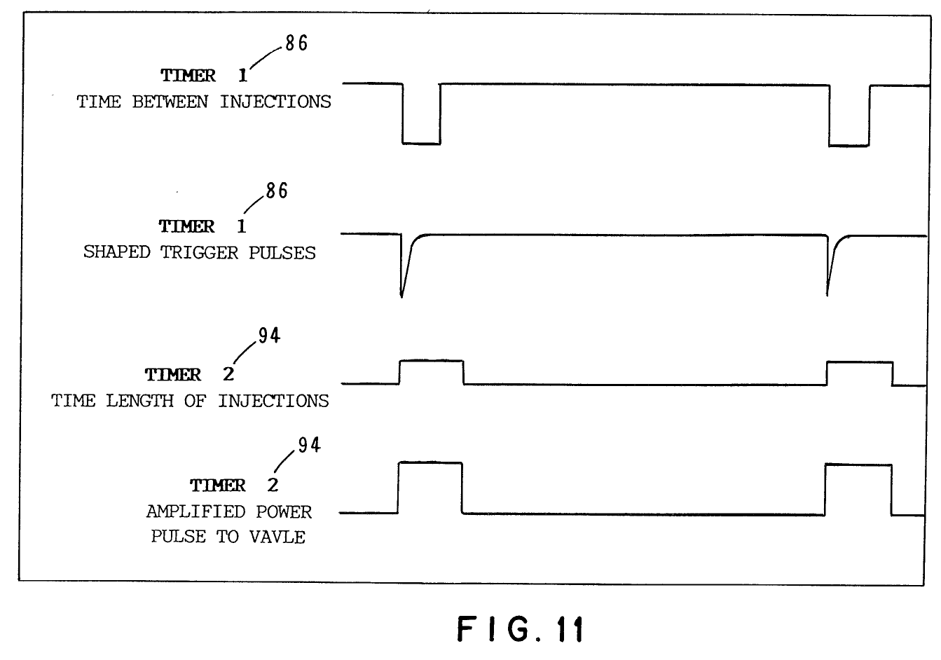

As best seen in FIGS. 7 and 10, the electrically operated flow control means here comprises an electrically operated solenoid valve 86 and valve control means operably associated with the solenoid valve for controlling the frequency of opening the solenoid valve. Solenoid valve is of conventional construction as is readily commercially available from sources such as the Lee Company of Westbrook, Conn.

As indicated in FIG. 10, the valve control means comprises a control circuit generally designated by the numeral 88 that includes a pair of complementary metal oxide semiconductors 90 and 92. In a manner presently to be described, a first rotatable control assembly 94, which is operably associated with solenoid valve 86 functions to set a time for opening the valve. Similarly, a second rotatable control assembly 96, which is also operably associated with solenoid valve 86 functions to set the time interval between the opening and closing of the solenoid valve (see FIGS. 1, 7, 8, and 9).

At the heart of control circuit 86 is the pair of previously mentioned complementary metal oxide semiconductors (CMOS) 90 and 92. These classic integrated circuit timer chips are highly stable devices that are readily commercially available from sources such as National Semi-Conductor Corporation of Santa Clara, Calif. and can be used as time-delay devices or as free-running muftivibrators. Details concerning these semi-conductors are available from National Semi-Conductor Corporation. In the time-delay configuration, the delay is precisely controlled by one external resistor 98 and one external capacitor 100. In the free-running mode, the frequency (inverse-period) and the duty cycle are precisely controlled by two external resistors 102 and 104 respectively and one external capacitor 106. The CMOS timers are used in the present application because they use substantially less battery power than other types of conventional bipolar devices. For example, in the present application, the power drain has been determined to be on the order of less than 1.5 milliwatts. Power is supplied in this instance by a conventional battery 107.

In operating the flow control means of the present invention, when the circuit shown in FIG. 10 is energized, the free running oscillator, or CMOS 90 will run with a period determined precisely by resistors 102 and 104. Capacitor 106 will remain fixed over the entire range. The period-setting resistors 102 and 104 form a part of the previously identified control assembly or six-sided plug-in unit 94 having a rotatable control knob 84a, (FIG. 8). Each six-sided control unit provides six discrete timing periods between infusions of pharmaceutical product as, for example, 15 minutes, 30 minutes, one hour, two hours, four hours and eight hours. Where desired, as many as four six-sided plug-in units can be used with each unit being color-coded or otherwise marked so as to provide a total of 24 individual (discrete) periods between Infusions. It is to be understood that these time periods, which can be easily set by the caregiver or physician, can vary from application to application and the time periods identified are exemplary only.

The time delay means that comprises CMOS 92 is designed to have a preset “on” time that is precisely determined by resistor 98. In the present instance, capacitor 100 will remain fixed over the entire dosage range. The setting resistor 98 is contained within the previously identified rotatable control assembly, or four sided plug-in unit 96 shown in FIG. 9. The desired dosage can be selected by simply rotating the knob 96a of the four-sided, plug-in 96 to the selected number imprinted on the base of the device. Dosage levels as low as 10 micro-liters can be dispensed because the valve can open and close in milliseconds. By way of non-limiting example, the dosage levels can range from 10 micro-liters to 20 micro-liters to 40 micro-liters to 80 micro-liters. In this regard, it is to be understood that, since the pressure exerted on the medicinal fluid contained within reservoir 22 by distendable membrane 28 is substantially constant, precise control of open valve time will insure uniform dosages.

In using the apparatus of this form of the invention, tear-away cap 78 is removed from the syringe assembly 40. This done, cap 76 is similarly removed from the connector subassembly 36 of the fluid dispenser so that the syringe assembly can be mated with the connector subassembly in the manner illustrated in FIG. 5. As shown in FIG. 2, the barrel or polarity collar 60 of the syringe assembly is provided with a plurality of circumferentially spaced, key-like protuberances 60a. In a bayonet-like fashion, these key-like protuberances are receivable within key-ways or slots 37 formed in connector subassembly 36 and the syringe assembly is rotated to secure the connection in a manner well understood by those skilled in the art. It is to be understood that the polarity of collar 60 could be provided with any number of protuberances, as for example, two or four and the connector subassembly 36 could similarly be provided with any number of key-ways or slots. In this way only properly configured syringe assemblies could be mated with a given fluid delivery component.

As the syringe is mated with the connector subassembly, cannula 58 will pierce septum 64 in the manner illustrated in FIG. 5. With the components of the apparatus thusly mated in the manner shown in FIG. 5, a force exerted on the pusher hub 70a of the syringe assembly will cause fluid to flow through the cannula, past the umbrella check valve 50, and into reservoir 22. As the fluid flows into reservoir 22, distendable membrane 28 will distend from the configuration shown in FIG. 5 to the configuration indicated by the phantom lines in FIG. 5.

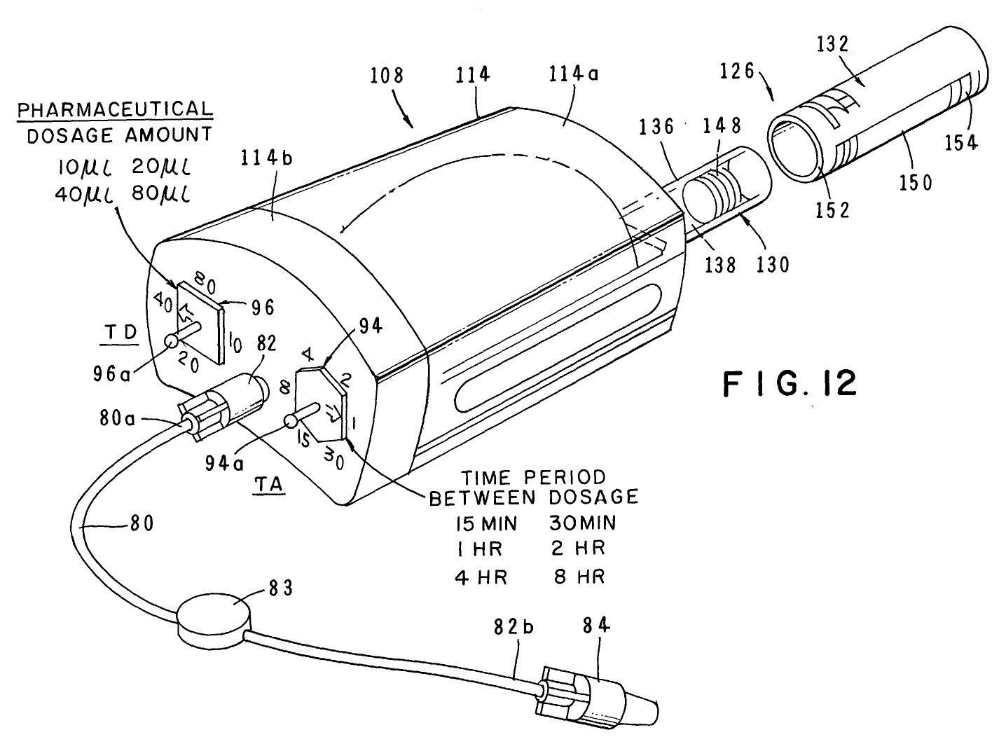

With the reservoir filled, fluid can be dispensed from the fluid dispenser by operating the novel flow control means of the invention. As previously mentioned, this is initially accomplished by the selective rotation of the rotatable knobs 94a and 96a of the plug in controls 94 and 96 respectively. More particularly, rotation of control knob 94a will set one of six discrete timing periods between the infusions of the medicament or pharmaceutical product. For example, control knob 94 could be rotated to an intermediate position providing for an infusion period of one hour. Similarly, control knob 96a of plug-in 96 could be rotated to an intermediate dosage level of, for example, 40 micro liters (see also FIG. 12). In the manner previously described, the control circuitry operably associated with the plug-in units 94 and 96 will appropriately operate the solenoid valve 86 in a manner to open the valve at an appropriate time and cause the valve to remain open for an appropriate period of time in the manner schematically depicted in FIGS. 10 and 11. More particularly, as shown in FIG. 11, operation of the control knob 96 will set the time between injections, in this case one hour, and control unit 94 will set the time length of injections, in this case to provide a dosage level of 40 micro liters. As previously mentioned, since the pressure exerted on the medicinal fluid contained within reservoir 22 by the distendable membrane 28 is substantially constant, precise control of the time during which the solenoid is open will ensure the delivery of dosages of uniform volume to the patient. In this regard, it can be seen, by referring to FIG. 5, as the distendable membrane moves toward its starting position fluid will flow outwardly of outlet 53, into passageway 54 and then into passageway 56. From passageway 56, the medicinal fluid will flow through the filter means, or filter assembly 85 and then toward the solenoid valve 86. So long as the solenoid valve is open, fluid will flow at a constant rate toward the infusion means in the manner indicated by the arrows in FIG. 5.

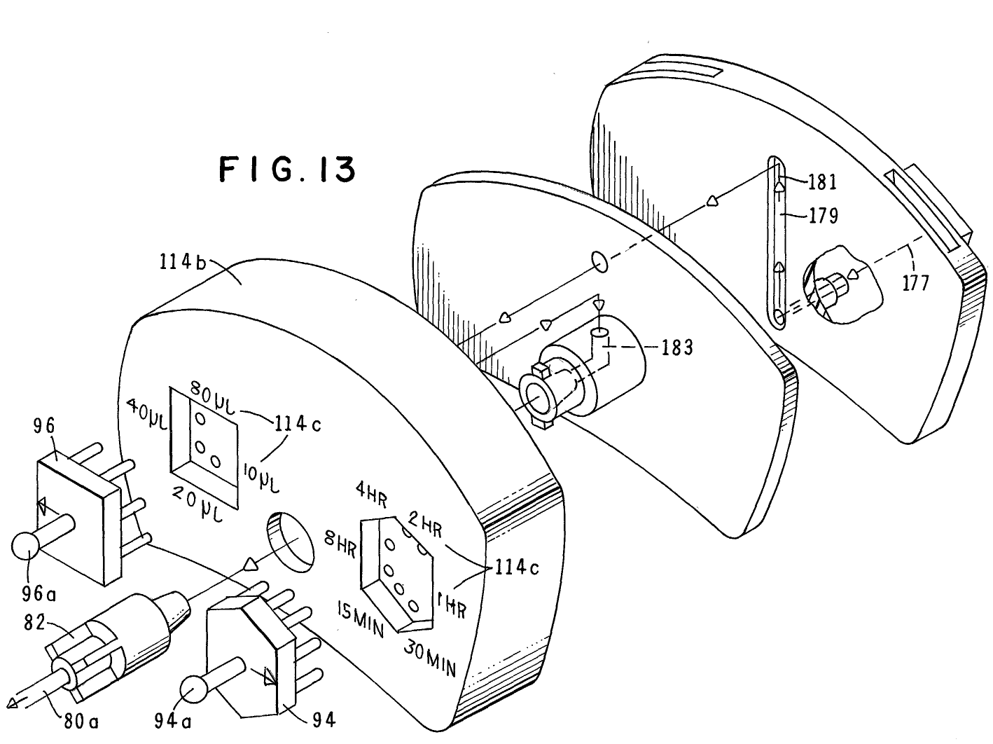

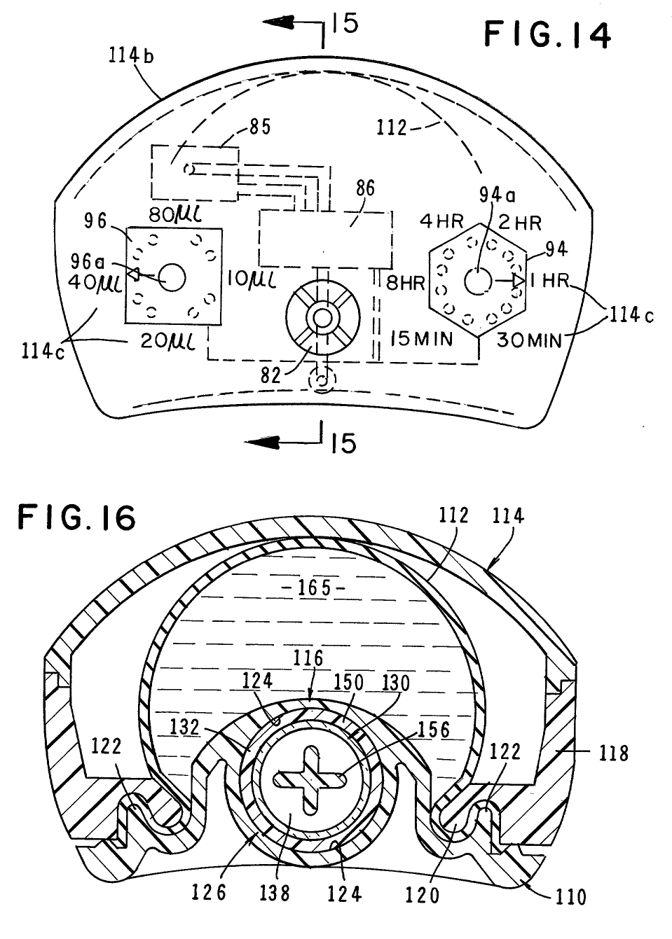

Referring next to FIGS. 12 through 18, an alternate embodiment of the invention is there shown in generally designated by the numeral 108. The fluid dispenser of this latter form of the invention is similar in many respects to that shown in FIGS. 1 through 4 and like numerals are used to identify like components. However, the fill means of this latest form of the invention, the character of which will presently be described, is substantially different.

Considering first the dispensing component of the invention, this component here comprises a base assembly 110, a stored energy source, shown here as a distendable membrane 112, and a cover 114 for enclosing the stored energy source. As before, cover 114 includes a rearward portion 114a and a forward cup-like housing 114b. Similarly, base assembly includes an ullage substrate 116 and a membrane capture housing 118 having a bottom opening 120 that receives the distendable membrane engaging element or protuberance 122 (see FIG. 15) of base assembly 110. Referring particularly to FIG. 15, the ullage substrate 116 is provided with fill assembly receiving means shown here as a longitudinally extending, generally cylindrically shaped receiving chamber 124 for receiving the fill assembly 126. Provided within chamber 126 are the valve and cannula means of the invention, the nature and purpose of which will presently be discussed.

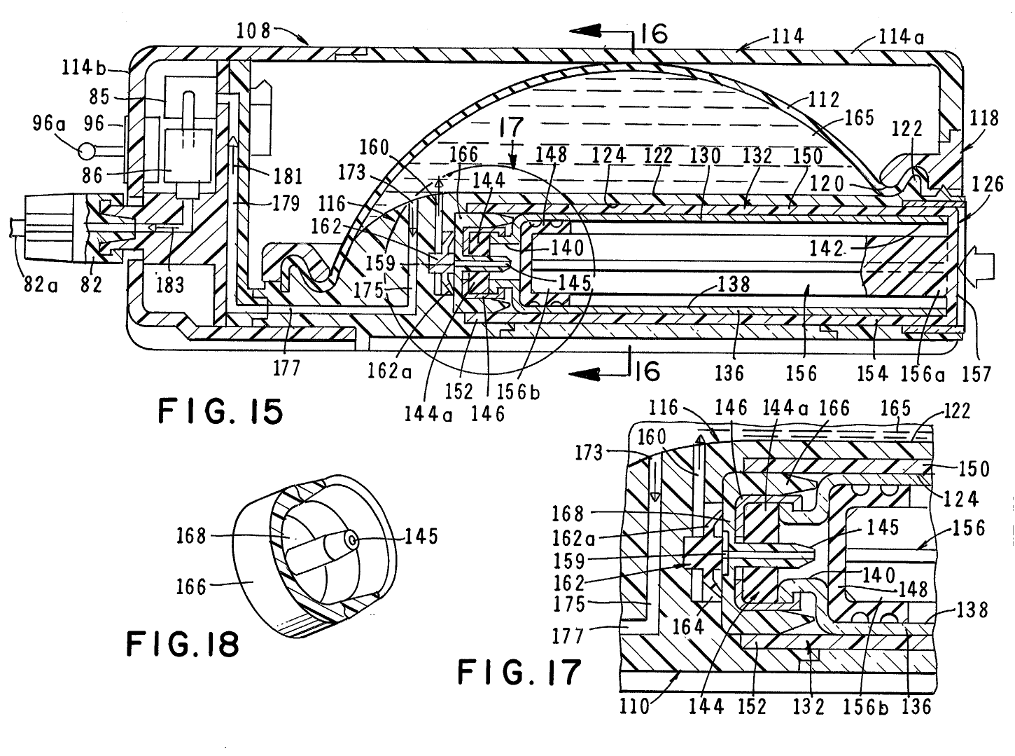

As best seen by referring to FIGS. 12 and 15, one form of the fill assembly 126 of the apparatus comprises a container subassembly 130 and an adapter subassembly 132. Container subassembly 130 includes a body portion 136, having a fluid chamber 138 for containing an injectable fluid. Chamber 138 is provided with first and second open ends 140 and 142 (FIG. 15). First open end 140 is sealably closed by closure means here provided in the form of a pierceable septum assembly 144 that includes a septum 144a. Septum 144a is pierceable by the cannula means of the invention that is shown in FIGS. 15 and 18 as a blunt end, hollow cannula 145. Septum assembly 144 is held securely in position within open end 140 by clamping ring 146. As best seen in FIGS. 12, 15 and 17, to expel fluid from chamber 138, a plunger 148 is telescopically movable within the chamber from a first location where it is proximate second open end 142 to a second position shown in FIG. 15 where it is proximate first open end 140. The vial or body portion of the container subassembly 130 can be constructed of various materials such as glass and plastic.

Referring particularly to FIGS. 12 and 15, it can be seen that the adapter subassembly 132 of this form of the invention comprises a hollow housing 150 having a first open end 152 and a second closed end 154. Container subassembly 130 is telescopically receivable within open end 152 of housing 150 in the manner shown in the drawings so that the housing can be moved from the first extended position shown in FIG. 12 to the vial encapsulation position shown in FIG. 15. Forming an important part of the adapter subassembly is pusher means shown here as an elongated pusher rod 156 which functions to move plunger 148 within fluid chamber 138 from the first position to a second position shown in FIG. 15. In the form of the invention shown in the drawings, pusher rod 156 has a first end 156a that is interconnected with closure wall 157 and an opposite end 156b that engages plunger 148 and causes telescopic movement of the plunger within chamber 138 of container subassembly 130 as housing 150 is moved from the extended position shown in FIG. 12 into the vial encapsulating position shown in FIG. 15.

As plunger 148 is moved forwardly of container 130 by the insertion of the fill assembly into chamber 124, the fluid contained in the container will flow under pressure into a fluid inlet 159 formed in base 110 (FIG. 5) and then into a passageway 160 via the valve means which is here provided as an umbrella type check valve 162. As best seen in FIG. 17, valve 162, which is of a conventional construction, is received within a cavity 164 formed in the end wall of receiving chamber 124 and is held in position therewithin by a cylindrically shaped housing 166 having an end wall 168 which supports cannula 185 in the manner shown in FIG. 4. Valve 162 is constructed from an appropriate elastomer and has a resiliently deformable skirt portion 162a that will deform inwardly within cavity 164 to permit fluid to flow toward the reservoir of the device, but will block reverse flow. From passageway 160, the fluid will flow under pressure into reservoir 165 where it will cause the stored energy means or membrane 112 to distend outwardly from protuberance 122 of ullage substrate 116 in the manner shown in FIG. 15.

Turning to FIG. 12, the infusion means of this latest form of the invention is similar to that previously described and can be seen to comprise an elongated fluid delivery line 80 having a first end 80a connected to the outlet port connector fitting 82 of forward portion 114b of the fluid dispenser and a second end 82b to which a conventional luer connector assembly 84 is connected. Disposed intermediate the first and second ends of the fluid delivery line is a conventional gas vent assembly 86.

As in the earlier described embodiment, the infusion means of the invention also includes filter means for filtering particulate matter from the medicinal fluids flowing toward the patient. The filter means comprises a conventional filter unit 85 of a character previously described. As shown in FIG. 15, filter assembly 85 is housing within the forward portion 114b of the dispenser housing. Also housed within forward portion 114b is the electrically operated flow control means of the invention which functions to precisely control the flow of medicinal fluid toward the patient. This flow control means is identified in construction and operation to that previously described and functions to control both the frequency of the flow of medicinal fluid toward the patient as well as the volume of medicinal fluid flowing toward the patient. Accordingly, reference should be made to FIGS. 7, 10 and 111 that illustrate the construction of the electrically operated flow control means of the invention.

In using this latest form of the apparatus of the invention, the outboard end of the container assemblage is inserted within the open end of the adapter subassembly 132 and the assemblage thus formed is, in turn, inserted into chamber 124 of the dispensing component. An inward pressure exerted on the adapter assembly in the direction of the arrow of FIG. 15 will cause cannula 145 to pierce the septum 144a of the container assembly. A continued inward pressure on the adapter subassembly will cause pusher rod 156 to move plunger 148 inwardly of chamber 138 of the container subassembly causing the fluid contained within the container to flow through the cannula, past the umbrella check valve 162 and into reservoir 165. As the fluid flows into reservoir 165, distendable membrane 112 will distend into the position shown in FIG. 15.

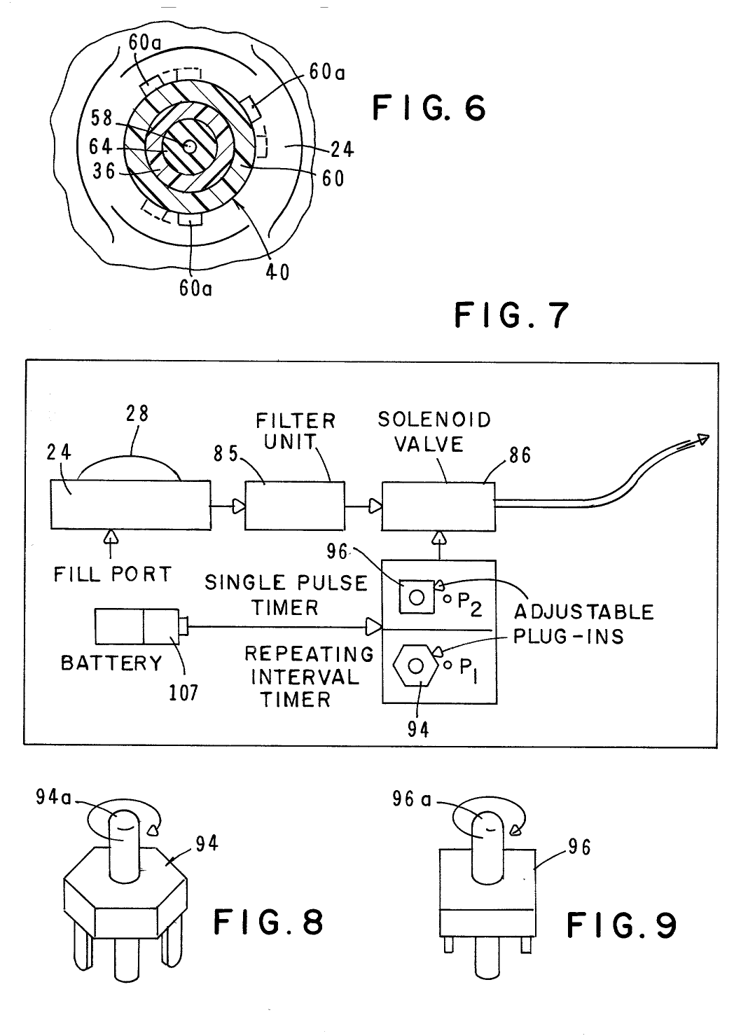

With the reservoir filled, fluid can be dispensed from the fluid dispenser by operating the flow control means of the invention in the manner described in connection with the embodiment of the invention shown in FIGS. 1 through 11. As previously mentioned, this is initially accomplished by the selective rotation of the rotatable knobs 94a and 96a of the plug in controls 94 and 96 respectively. As indicated in FIG. 12, rotation of the control knob 94a will set one of six discrete timing periods identified by indicia 114c on the face of the device between the infusions of the medicament or pharmaceutical product. For example, control knob 94 is here rotated to an intermediate position providing for an infusion period of one hour. Similarly, control knob 96a of plug-in 96 is here rotated to an intermediate dosage level of 10 micro liters.

As earlier described, the control circuitry operably associated with the plug-in units 94 and 96 will appropriately operate the solenoid valve 86 in a manner to open the valve at an appropriate time and will cause the valve to remain open for an appropriate period of time in the manner schematically depicted in FIGS. 10 and 11. As depicted in FIG. 11, operation of the control knob 96 will set the will set the time between injections, in this case one hour, and control unit 94 will set the time length of injections, in this case to provide a dosage level of 10 micro liters. Because in operation, the pressure exerted on the medicinal fluid contained within reservoir 165 by the distendable membrane 112 is substantially constant, precise control of the time during which the solenoid is open will ensure uniform dosage volume to the patient. During the fluid delivery mode, as distendable membrane moves toward its starting position, fluid will flow outwardly of outlet 173, into passageway 175 and then into passageway 177 (FIG. 15). From passageway 177, the medicinal fluid will flow into a passageway 179, through the filter means, or filter assembly 85, in the direction of the arrow 181 and then toward the solenoid valve 86. So long as the solenoid valve is open, fluid will flow at a constant rate toward the infusion means in the manner indicated by the arrow 183 in FIG. 15.

Having now described the invention in detail in accordance with the requirements of the patent statutes, those skilled in this art will have no difficulty in making changes and modifications in the individual parts or their relative assembly in order to meet specific requirements or conditions. Such changes and modifications may be made without departing from the scope and spirit of the invention, as set forth in the following claims.

I claim:

1. A device for use in infusing medicinal fluid into a patient at a controlled rate comprising:
(a) a fluid dispenser including:
(i) a base having a fluid inlet;
(ii) a stored energy means for forming, in conjunction with said base a fluid reservoir having an inlet in communication with said fluid inlet of said base and an outlet, said stored energy means comprising at least one distendable member superimposed over said base, said member being distendable as a result of pressure imparted by the fluids to be infused to establish internal stresses, said stresses tending to move said member toward a less distended configuration;
(iii) an outlet port in communication with said outlet of said fluid reservoir for dispensing fluids from the device; and
(b) electrically operated flow control means operably associated with said fluid dispenser and disposed between said outlet port of said fluid dispenser and the patient for controlling the flow of medicinal fluid toward the patient.

2. The apparatus as defined in claim 1 further including reservoir fill means interconnectable with said base of said fluid dispenser for filling said fluid reservoir thereof.

3. The apparatus as defined in claim 1 in which said electrically operated flow control means controls the frequency of the flow of medicinal fluid toward the patient.

4. The apparatus as defined in claim 1 in which said electrically operated flow control means controls the volume of medicinal fluid flowing toward the patient.

5. The apparatus as defined in claim 1 in which said electrically operated flow control means comprises a solenoid valve.

6. The apparatus as defined in claim 5 further including control means operably associated with said solenoid valve for controlling the opening and closing of said solenoid valve.

7. A device for use in infusing medicinal fluid into a patient at a controlled rate comprising:
(a) a fluid dispenser including:
(i) a base;
(ii) a stored energy means for forming, in conjunction with said base a fluid reservoir having an inlet and an outlet, said stored energy means comprises at least one, distendable elastomeric membrane superimposed over said base, said membrane being distendable as a result of pressure imparted by the fluids to be infused to establish internal stresses, said stresses tending to move said member toward a less distended configuration;
(iii) an outlet port in communication with said outlet of said fluid reservoir for dispensing fluids from the device;
(b) reservoir fill means interconnected with said fluid dispenser for filling said fluid reservoir thereof; and
(c) electrically operated flow control means operably associated with said fluid dispenser and disposed between said outlet port of said fluid dispenser and the patient for controlling the flow of medicinal fluid toward the patient, said electrically operated flow control means comprising an electrically operated solenoid valve.

8. The apparatus as defined in claim 7 further including filter means operably associated with said fluid dispenser and disposed between said outlet of said fluid dispenser and the patient for filtering the medicinal fluid flowing toward the patient.

9. The apparatus as defined in claim 7 in which said electrically operated flow control means controls the frequency of the flow of medicinal fluid toward the patient.

10. The apparatus as defined in claim 8 in which said electrically operated flow control means controls the volume of medicinal fluid flowing toward the patient.

11. The apparatus as defined in claim 9 further including control means operably associated with said solenoid valve for controlling the frequency of opening said solenoid valve.

12. The apparatus as defined in claim 10 in which said control means comprises a control circuit including a complementary metal oxide semiconductor.

13. The apparatus as defined in claim 11 in which said control means further comprises a first rotatable control knob operably associated with said control circuit for setting a time for opening said solenoid valve.

14. The apparatus as defined in claim 13 in which said control means further comprises a second rotatable control knob operably associated with said control circuit for setting the time interval between the opening and closing of said solenoid valve.

15. A device for use in infusing medicinal fluid into a patient at a controlled rate comprising:
(a) a fluid dispenser including:
(i) a base having, an upper surface and a lower surface;
(ii) a stored energy means for forming, in conjunction with said base a fluid reservoir having an inlet and an outlet, said stored energy means comprises at least one, distendable elastomeric membrane superimposed over said base, said membrane being distendable as a result of pressure imparted by the fluids to be infused to establish internal stresses, said stresses tending to move said member toward a less distended configuration;
(iii) an outlet port in communication with said outlet of said fluid reservoir for dispensing fluids from the device;
(b) reservoir fill means interconnectable with said fluid dispenser for filling said fluid reservoir thereof; and
(c) infusion means for delivering the medicinal fluid to the patient comprising:
(i) a delivery line connected to said outlet port of said housing for delivering fluid to the patient; and
(ii) electrically operated flow control means disposed within said delivery line between said outlet port of said fluid dispenser and the patient for controlling the flow of medicinal fluid toward the patient, said electrically operated flow control means comprising an electrically operated solenoid valve for controlling both the frequency of the flow of medicinal fluid toward the patient and the volume of medicinal fluid flowing toward the patient.

16. The apparatus as defined in claim 15 in which said reservoir fill means comprises a syringe.

17. The apparatus as defined in claim 15 in which said reservoir fill means comprises:
(a) a container subassembly including a container having a body portion, a fluid chamber, and first and second open ends; closure means for sealably closing said first open end of said container; and a plunger telescopically movable within said container from a first location proximate said second open end to a second, spaced-apart location; and
(b) an adapter subassembly comprising a hollow housing having a first open end for telescopically receiving a part of said body portion of said container of said container subassembly and pusher means for engagement with said plunger of said container subassembly to move said plunger within said container between said first and second locations.

18. The apparatus as defined in claim 15 further including filter means operably associated with said fluid dispenser and disposed between said outlet of said fluid dispenser and the patient for filtering the medicinal fluid flowing toward the patient.

19. The apparatus as defined in claim 15 in which said control means comprises a control circuit including a complementary metal oxide semiconductor.

20. The apparatus as defined in claim 11 in which said control means further comprises a first rotatable control knob operably associated with said control circuit for setting a time for opening said solenoid and a second rotatable control knob operably associated with said control circuit for setting the time interval between the opening and closing of said solenoid valve.
